(19) United States
(12) Patent Application Publication
(10) Pub. No.: US 2003/0059888 A1
(43) Pub. Date: Mar. 27, 2003

(54) Compositions and methods for generating antigen-binding units

(76) Inventor: Shengfeng Li, Belmont, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/226,950

(22) Filed: Aug. 22, 2002

Related U.S. Application Data
(60) Provisional application No. 60/314,478, filed on Aug. 22, 2001.

(51) Int. Cl. C12P 21/02; C12N 1/21; C12N 15/74; C12N 15/85; C12N 5/06

(52) U.S. Cl. 435/69.1; 435/326; 435/455; 435/472; 435/252.3; 435/320.1

(57) ABSTRACT

The present invention provides vectors that encode single-chain antigen-binding units in both prokaryotic and eukaryotic cells. The vectors are particularly useful for generating a genetically diverse repertoire of single-chain antigen-binding units to facilitate an in vivo screening of antigen-binding units that bind to a desired antigen inside a cell. The present invention also provides recombinant polynucleotides, host cells and kits comprising the vectors. Further provided by the invention are methods of using the subject vectors.

### CROSS-REFERENCE TO RELATED APPLICATIONS

This application claims the priority benefit of U.S. Provisional Patent Application No. 60/314,478, filed Aug. 22, 2001, pending, which is hereby incorporated herein by reference in its entirety.

### TECHNICAL FIELD

This invention is in the field of immunology. Specifically, the invention relates to the construction of vectors encoding single-chain antigen-binding units in both prokaryotic and eukaryotic cells. The compositions and methods embodied in the present invention are particularly useful for generating a genetically diverse repertoire of single-chain antigen-binding units to facilitate an in vivo screening of antigen-binding units that bind to a desired antigen inside a cell.

### BACKGROUND OF THE INVENTION

The immune response of a vertebrate provides a protective system that distinguishes foreign entities from native entities. Immune responses are the primary responsibilities of the B and T lymphocytes, which mediate the humoral response and the cell-mediated response, respectively. The humoral response is elicited by the B-cells which secrete antibodies (also known as immunoglobulins). Antibodies or immunoglobulins are molecules that recognize and bind to specific cognate antigens. Because of their exclusive specificities, antibodies, particularly monoclonal antibodies, are essential tools for analyzing the functions of biological molecules. Antibodies can be used to detect the protein expression levels, identify the protein-protein interaction complexes, localize the cellular compartment and tissue specificity, and analyze gene functions by neutralizing the gene product. Furthermore, antibodies have been widely used in the diagnosis and treatment of a variety of human diseases.

The basic immunoglobulin (Ig) in vertebrate systems is composed of two identical light (“L”) chain polypeptides (approximately 23 kDa), and two identical heavy (“H”) chain polypeptides (approximately 53 to 70 kDa). The four chains are joined by disulfide bonds in a “Y” configuration. At the base of the Y, the two H chains are bound by covalent disulfide linkages. The L and H chains are organized in a series of domains. The L chain has two domains, corresponding to the C region (“CL”) and the other to the V region (“VL”). The H chain has four domains, one corresponding to the V region (“VH”) and three domains (CH1, CH2 and CH3) in the C region. The antibody contains two arms (each arm being a Fab fragment), each of which has a VL and a VH region associated with each other. It is this pair of V regions (VL and VH) that differ, from one antibody to another (due to amino acid sequence variations), and which together are responsible for recognizing the antigen and providing an antigen-binding site. More specifically, each V region is made up from three complementarity determining regions (CDR) separated by four framework regions (FR). The CDR's are the most variable part of the variable regions, and they perform the critical antigen binding function. The CDR regions are derived from many potential germ line sequences via a complex process involving recombination, mutation and selection.

Research in recent years has demonstrated that the function of a binding antigen can be performed by fragments of a whole antibody. For instance, certain single-chain antigen-binding units containing the VL and VH regions fused together as a monomeric polypeptide have been shown to bind their corresponding antigens (Bird et al. (1988) Science 242:423-426 and Huston et al. (1988) PNAS 85:5879-5883). However, it is a well known problem in the art that not all antibodies can be made as single chains and still retain high binding affinity (Huston et al. (1988) Proc. Natl. Acad. Sci. U.S.A. 85:5879-5883; Stemmer et al. (1993) Biotechniques 14(2): 256-265). In part, this is due to the interference of linker sequences with the antigen binding sites. Furthermore, the propensity of single-chain antigen-binding units to aggregate inside a cell also hampers their intracellular antigen-binding capabilities.

To efficiently isolate those single-chain antigen-binding units with the desired intracellular binding capabilities, a vast diverse repertoire of distinct single-chain antibody molecules must be generated that are amenable to in vivo selection.

WO 00/54057 describes the use of a well-established two-hybrid system to detect the specific binding of a single-chain antigen-binding unit to its cognate antigen inside a yeast cell. The PCT publication does not describe or even suggest a method of constructing a diverse repertoire of single-chain antigen-binding units that allow the isolation of desired single-chain antigen-binding units using a two-hybrid system.

U.S. Pat. No. 5,733,743 teaches the use of a site-specific recombination sequence for constructing phage display libraries. Specifically, this patent describes loxP sequences for antibody chain recombination to derive a large repertoire of antigen-binding units that are displayed by phage particles. This patent does not teach or suggest a way of generating antigen-binding units with desired intracellular binding capabilities. It also does not teach any intracellular screening method, such as the one involving a two-hybrid system.

Thus, there remains a need for improved compositions and methods to generate a diverse repertoire of single-chain antigen-binding units that are amenable to in vivo screening of molecules capable of binding to their respective antigens within a cell. The present invention satisfies these needs and provides related advantages as well.

### SUMMARY OF THE INVENTION

A central aspect of the present invention is the design of a vector suited for generating antigen-binding units in both prokaryotic and eukaryotic cells. The vectors of the present invention are particularly useful for generating a genetically diverse repertoire of single-chain antigen-binding units to facilitate an in vivo screening of binding units that bind to a desired antigen inside a cell. Antigen-binding units capable of binding to their respective antigens (i.e. “intracellular” antigen-binding units) inside a cell are of tremendous research and therapeutic value. The ability of these binding units to specifically inhibit a protein's function and/or expression allows one to elucidate the biological function of the protein by creating, essentially, a protein-specific “knock-out” cell. Thus, the generation of these antibodies facilitate functional genomics studies.

Accordingly, in one embodiment, the present invention provides a vector replicable in both prokaryotic and eukaryotic cells. The vector comprises a polynucleotide encoding a single-chain antigen-binding unit. The polynucleotide comprises: (a) a variable region of a first antibody chain; (b) a first site-specific recombination sequence; (c) a variable region of a second antibody chain; and (d) a second site-specific recombination sequence. The two site-specific recombination sequences facilitate recombination of the variable regions of (a) and (c) between two compatible vectors.

In another embodiment, the invention provides a vector replicable in both prokaryotic and eukaryotic cells. The vector comprises a polynucleotide encoding a single-chain antigen-binding unit fused to a gene activation moiety. The polynucleotide comprises: (a) a variable region of a first antibody chain; (b) a first site-specific recombination sequence; (c) a variable region of a second antibody chain fused to a gene activation moiety region; and (d) a second site-specific recombination sequence. The two site-specific recombination sequences facilitate recombination of the variable regions of (a) and (c) between two compatible vectors, and wherein the gene activation moiety facilitates detection of specific binding to an antigen in a eukaryotic cell.

In one aspect of these embodiment, the first antibody chain contained in the vector is a light chain and the second antibody chain is a heavy chain, or vise versa. The light or heavy chain may comprise human or non-human sequences. The two site-specific recombination sequences may be the same or they may be of different sequences. Preferred recombination sites are sequences derived from Frt and loxP. LoxP sites include but are not limited to loxP2 and loxP511.

The vector may further comprise at least two origins of replication, wherein at least one first origin facilitates replication in a prokaryotic cell, and at least one second origin facilitates replication in a eukaryotic cell. Representative prokaryotic cells are bacterial cells such as E. coli, and exemplary eukaryotic cells are yeast cells including but not limited to S. cerevisiae.

In certain embodiments, the vectors contain a gene activation moiety comprising a transcription activation domain of a protein selected from the group consisting of GAL4 and VP16. Such a moiety facilitates the detection of specific binding to a desired antigen intracellularly by employing, e.g., a two-hybrid system.

This invention further provides a library of the subject vectors and host cells comprising the subject vectors.

Also included in the present invention is a method of generating a selectable library of vectors encoding a genetically diverse repertoire of single-chain antigen-binding units. The method involves the steps of: (a) providing a plurality of the subject vectors; and (b) causing or allowing site-specific recombination of the variable regions encoded by at least two compatible vectors, thereby generating the selectable library. In one aspect, the recombination occurs in vitro in the presence of a site-specific recombinase. In another aspect, the recombination occurs in a cell expressing a site-specific recombinase. In the case of in vivo recombination, the method may further involve the steps of (a) introducing a plurality of the vectors into a population of prokaryotic cells; (b) infecting a first population of prokaryotic cells with a plurality of helper phages to yield a population of phage particles; (c) infecting a second population of prokaryotic cells with the phage particles of (b); and optionally repeating the step of (c), thereby introducing a plurality of the vectors into a cell. Such steps may employ helper phages such as M13 helper phages. The recombined repertoire has a complexity ranging from about 10⁶ to about 10¹³, and preferably from about 10⁷ to about 10⁹. A more preferred range is from about 10⁸ to about 10¹⁰, and more preferably from about 10⁸ to about 10¹¹. Even more preferred is a range from about 10⁹ to about 10¹⁰, and yet even more preferably from about 10⁹ to about 10¹¹. The recombinase employed preferably is Cre-recombinase.

Further encompassed in the present invention is a selectable library of vectors generated by the aforementioned method. The host cells including yeast cells harboring the selectable library are also contemplated. Finally, the present invention provides a kit comprising the subject vectors in suitable packaging.

### EXPLAINATION OF ABBREVIATIONS USED HEREIN

1. Nsc: Non-single chain

2. Sc: Sing-chain

3. Abu: Antigen-binding unit

4. Abus: Antigen-binding units

4. L chain: Light chain

5. H chain: Heavy chain

6. VL: Light chain variable region

7. VH: Heavy chain variable region

### BRIEF DESCRIPTION OF THE DRAWINGS

FIG. 1 is a schematic representation of the plasmid designated pSF90. The vector encodes a single-chain antigen-binding unit in which the VL and H region are linked by loxP and loxP2 sites. The loxP2 site is a mutant loxP sequence with two point mutations. The single chain is fused with VP16 transcription activation domain. A Flag tag is also added to the C-terminus. The wildtype loxP sequence is placed down stream of the single-chain coding sequences.

FIG. 2 is a schematic representation of the plasmid designated pSF83. The vector encodes the antigen, Ras, which was used for screening Ras-binding single-chain antigen-binding units using a two-hybrid system.

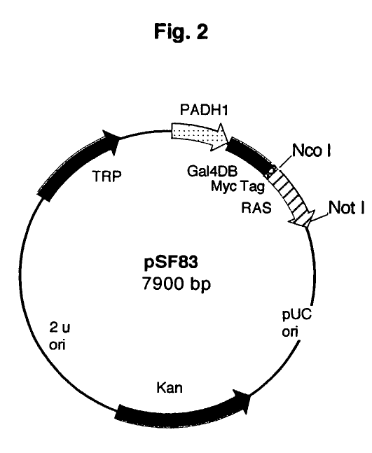

FIG. 3 depicts a recombination scheme of VH and VL regions using site-specific recombination sites. The recombination is exponential.

FIG. 4 shows the specific binding of an anti-Ras binding unit to its respective antigen Ras.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference in their entirety into the present disclosure.

General Techniques:

The practice of the present invention will employ, unless otherwise indicated, conventional techniques of immunology, biochemistry, chemistry, molecular biology, microbiology, cell biology, genomics and recombinant DNA, which are within the skill of the art. See, e.g., Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

Definitions:

The terms “polypeptide”, “peptide” and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptides have an amino acid sequence that is essentially identical to that of a polypeptide encoded in the sequence, or a portion thereof. Preferably, the portion consists of at least 10-20 amino acids, and more preferably at least 20-30 amino acids. Most preferred would be a portion of at least 30-50 amino acids, or a portion which is immunologically identifiable with a polypeptide encoded in the sequence. This terminology also includes a polypeptide expressed from a designated nucleic acid sequence.

A “chimeric” or “hybrid” protein contains at least one fusion polypeptide comprising regions in a different position in the sequence than what occurs in nature. The regions may normally exist in separate proteins and are brought together in the fusion polypeptide; or they may normally exist in the same protein but are placed in a new arrangement in the fusion polypeptide. A chimeric or hybrid protein may be created, for example, by chemical synthesis, or by creating and translating a polynucleotide in which the peptide regions are encoded in the desired relationship.

A “multimeric protein” as used herein refers to a globular protein containing more than one separate polypeptide or protein chain associated with each other to form a single globular protein in vitro or in vivo. The multimeric protein may consist of more than one polypeptide of the same kind to form a “homomultimer.” Alternatively, the multimeric protein may also be composed of more than one polypeptide of distinct sequences to form a “heteromultimer.” Thus, a “heteromultimer” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where more than two polypeptides are present. Exemplary structures for the heteromultimer include heterodimers (e.g. Fab fragments, diabodies, Fv fragments dimerized via the interaction of a first and second leucine zipper,) trimeric G-proteins, heterotetramers (e.g. F(ab′)₂ fragments) and further oligomeric structures.

The term “antibody” as used herein refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally-occurring antibodies. These fragments are collectively termed “antigen-binding units” (“Abus”). Abus can be broadly divided into “single-chain” (“Sc”) and “non-single-chain” (“Nsc”) types based on their molecular structures. The terms “the first” or “the second” antibody chain as applied to an antigen-binding unit refers the light or the heavy antibody chain.

Also encompassed within the terms “antibodies” and “Abus” are immunoglobulin molecules of a variety of species origins including invertebrates and vertebrates. The term “human” as applied to an antibody or an Abu refers to an immunoglobulin molecule expressed by a human gene or fragment thereof. The term “humanized” as applied to non-human (e.g. rodent or primate) antibodies are hybrid immunoglobulins, immunoglobulin chains or fragments thereof which contain minimal sequences derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In general, the humanized antibody will comprise substantially all of at least one, and typically two, variable domains, in which all or substantially all of the CDR regions correspond to those of a non-human immunoglobulin. Moreover, all or substantially all of the FR regions are those of a human immunoglobulin sequence. The humanized antibody may also comprise at least a portion of an immunoglobulin constant region (Fc), typically that of a human immunoglobulin.

As used herein, a “non-single-chain antigen-binding unit” (“Nsc Abu”) refers to a heteromultimer comprising a light-chain polypeptide and a heavy-chain polypeptide. “Light-chain polypeptide” means that the polypeptide contains sequences derived from a light chain of an immunoglobulin. Likewise, “heavy-chain polypeptide” means that the polypeptide contains sequences derived from a heavy chain of an immunoglobulin.

As noted above, a Nsc Abu can be either “monovalent” or “multivalent.” Whereas the former has one binding site per antigen-binding unit, the latter contains multiple binding sites capable of binding to more than one antigen of the same or of a different kind. Depending on the number of binding sites, a Nsc Abu may be bivalent (having two antigen-binding sites), trivalent (having three antigen-binding sites), tetravalent (having four antigen-binding sites), and so on.

Multivalent Nsc Abus can be further classified on the basis of their binding specificities. A “monospecific” Nsc Abu is a molecule capable of binding to one or more antigens of the same kind. A “multispecific” Nsc Abu is a molecule having binding specificities for at least two different antigens. While such molecules normally will only bind two distinct antigens (i.e. bispecific Abus), antibodies with additional specificities such as trispecific antibodies are encompassed by this expression when used herein. Examples of bispecific antigen binding units include those with one arm directed against a tumor cell antigen and the other arm directed against a cytotoxic trigger molecule such as anti-FcγRI/anti-CD15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA 1, anti-CD3/anti-CD 19, anti-CD3/MoV18, anti-neural cell ahesion molecule (NCAM)/anti-CD3, anti-folate binding protein (FBP)/anti-CD3, anti-pan carcinoma associated antigen (AMOC-31)/anti-CD3; bispecific Abus with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); bispecific Abus which can be used as fibrinolytic agents such as anti-fibrin/anti-tissue plasminogen activator (tPA), anti-fibrin/anti-urokinase-type plasminogen activator (uPA); bispecific antigen-binding untis for targeting immune complexes to cell surface receptors such as anti-low density lipoprotein (LDL)/anti-Fc receptor (e.g. Fcγ RI, FcγRII or FcγRIII); bispecific Abus for use in therapy of infectious diseases such as anti-CD3/anti-herpes simplex virus (HSV), anti-T-cell receptor:CD3 complex/anti-influenza, anti-FcγR/anti-HIV; bispecific Abus for tumor detection in vitro or in vivo such as anti-CEA/anti-EOTUBE, anti-CEA/anti-DPTA, anti-p185^{HER2}/anti-hapten; BsAbs as vaccine adjuvants (see Fanger et al., supra); and bispecific Abus as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase (see Nolan et al., supra). Examples of trispecific antibodies include anti-CD3/anti-CD4/anti-CD37, anti-CD3/anti-CD5/anti-CD37 and anti-CD3/anti-CD8/anti-CD37.

As used herein, a “single-chain antigen-binding unit” (“Sc Abu”) refers to a monomeric Abu. Although the two domains of the Fv fragment are coded for by separate genes, a synthetic linker can be made that enables them to be made as a single protein chain (i.e. single chain Fv (“scFv”) as described in Bird et al. (1988) Science 242:423-426 and Huston et al. (1988) PNAS 85:5879-5883) by recombinant methods. A preferred single-chain antigen-binding unit contains VL and VH regions that are fused together and stabilized by a site-specific recombination sequence including but not limited to loxP site. The scFvs can be assembled in any order, for example, VH—(first site-specific recombination sequence)—VL—(second site-specific recombination sequence), or VL—(first site-specific recombination sequence)—VH—(site-specific recombination sequence).

A “repertoire of antigen-binding units” refers to a plurality of antigen-binding units, at least two of which exhibit distinct binding specificities. A genetically diverse repertoire of antigen-binding units refers to a plurality of antigen-binding units, the majority of, if not all, the antigen-binding units exhibiting unique binding specificities with respect to each other. A genetically diverse repertoire typically has a complexity of at least 10⁶ to 10¹³, preferably between 10⁷ to 10⁹, more preferably between 10⁸ to 10¹⁰, and even more preferably between 10⁸ to 10¹¹ distinct antigen-binding units.

An antibody or Abu “specifically binds to” or “is immunoreactive with” an antigen if it binds with greater affinity or avidity than it binds to other reference antigens including polypeptides or other substances.

The terms “intracellular binding capability” and “binds intracellularly” refers to the ability of antigen-binding units to bind their respective antigens within a cell.

“Antigen” as used herein means a substance that is recognized and bound specifically by an antibody. Antigens can include peptides, proteins, glycoproteins, polysaccharides and lipids; portions thereof and combinations thereof. For the class of proteinaceous antigens, the antigens may be membrane, cytosolic, nuclear or secreted peptides or proteins.

As used herein, the term “surface antigens” refers to the plasma membrane components of a cell. Surface antigens encompass integral and peripheral membrane proteins, glycoproteins, polysaccharides and lipids that constitute the plasma membrane. An “integral membrane protein” is a transmembrane protein that extends across the lipid bilayer of the plasma membrane of a cell. A typical integral membrane protein consists of at least one “membrane spanning segment” that generally comprises hydrophobic amino acid residues. Peripheral membrane proteins do not extend into the hydrophobic interior of the lipid bilayer and they are bound to the membrane surface by noncovalent interaction with other membrane proteins.

The terms “membrane”, “cytosolic”, “nuclear” and “secreted” as applied to cellular proteins specify the extracellular and/or subcellular location in which the cellular protein is mostly, predominantly, or preferentially localized.

“Cell surface receptors” represent a subset of membrane proteins, capable of binding to their respective ligands. Cell surface receptors are molecules anchored on or inserted into the cell plasma membrane. They constitute a large family of proteins, glycoproteins, polysaccharides and lipids, which serve not only as structural constituents of the plasma membrane, but also as regulatory elements governing a variety of biological functions.

“Domain” refers to a portion of a protein that is physically or functionally distinguished from other portions of the protein or peptide. Physically-defined domains include those amino acid sequences that are exceptionally hydrophobic or hydrophilic, such as those sequences that are membrane-associated or cytoplasm-associated. Domains may also be defined by internal homologies that arise, for example, from gene duplication. Functionally-defined domains have a distinct biological function(s). The ligand-binding domain of a receptor, for example, is that domain that binds ligand. An antigen-binding domain refers to the part of an antigen-binding unit or an antibody that binds to the antigen. Functionally-defined domains need not be encoded by contiguous amino acid sequences. Functionally-defined domains may contain one or more physically-defined domains. Receptors, for example, are generally divided into the extracellular ligand-binding domain, a transmembrane domain, and an intracellular effector domain.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

A “cell line” or “cell culture” denotes bacterial, plant, insect or higher eukaryotic cells grown or maintained in vitro. The descendants of a cell may not be completely identical (either morphologically, genotypically, or phenotypically) to the parent cell.

A “defined medium” refers to a medium comprising nutritional and hormonal requirements necessary for the survival and/or growth of the cells in culture such that the components of the medium are known. Traditionally, the defined medium has been formulated by the addition of nutritional and growth factors necessary for growth and/or survival. Typically, the defined medium provides at least one component from one or more of the following categories: a) all essential amino acids, and usually the basic set of twenty amino acids plus cysteine; b) an energy source, usually in the form of a carbohydrate such as glucose; c) vitamins and/or other organic compounds required at low concentrations; d) free fatty acids; and e) trace elements, where trace elements are defined as inorganic compounds or naturally occurring elements that are typically required at very low concentrations, usually in the micromolar range. The defined medium may also optionally be supplemented with one or more components from any of the following categories: a) one or more mitogenic agents; b) salts and buffers as, for example, calcium, magnesium, and phosphate; c) nucleosides and bases such as, for example, adenosine and thymidine, hypoxanthine; and d) protein and tissue hydrolysates.

As used herein, the term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart.

Enrichment can be measured on an absolute basis, such as weight per volume of solution, or it can be measured in relation to a second, potentially interfering substance present in the source mixture. Increasing enrichments of the embodiments of this invention are increasingly more preferred. Thus, for example, a 2-fold enrichment is preferred, a 10-fold enrichment is more preferred, a 100-fold enrichment is more preferred, and a 1000-fold enrichment is even more preferred. A substance can also be provided in an isolated state by a process of artificial assembly, such as by chemical synthesis or recombinant expression.

“Linked,” “fused” or “fusion” are used interchangeably herein. These terms refer to the joining together of two more chemical elements or components, by whatever means, including chemical conjugation or recombinant means. An “in-frame fusion” refers to the joining of two or more open reading frames (OFRs) to form a continuous longer OFR, in a manner that maintains the correct reading frame of the original OFRs. Thus, the resulting recombinant fusion protein is a single protein containing two or more segments that correspond to polypeptides encoded by the original OFRs (which segments are not normally so joined in nature.) Although the reading frame is thus made continuous throughout the fused segments, the segments may be physically or spatially separated by, for example, an in-frame linker sequence (e.g. “flexon”), as described infra.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide which is known to comprise additional residues in one or both directions.

“Heterologous” means derived from a genotypically distinct entity from the rest of the entity to which it is being compared. For example, a promoter removed from its native coding sequence and operatively fused to a coding sequence other than the native sequence is a heterologous promoter. The term “heterologous” as applied to a polynucleotide, or a polypeptide, means that the polynucleotide or polypeptide is derived from a genotypically distinct entity from that of the rest of the entity to which it is being compared. For instance, a heterologous polynucleotide or antigen may be derived from a different species origin, different cell type, and the same type of cell of distinct individuals.

The terms “polynucleotides,” “nucleic acids,” “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Recombinant” as applied to a polynucleotide means that the polynucleotide is the product of various combinations of cloning, restriction and/or ligation steps, and other procedures that result in a construct that is distinct from a polynucleotide found in nature.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof.

“Operably fused” or “operatively fused” refers to a juxtaposition wherein the components so described are in a relationship permitting them to function in their intended manner. For instance, a promoter sequence is operably fused to a coding sequence if the promoter sequence promotes transcription of the coding sequence.

A “fusion gene” is a gene composed of at least two heterologous polynucleotides that are fused together.

A gene “database” denotes a set of stored data which represent a collection of sequences including nucleotide and peptide sequences, which in turn represent a collection of biological reference materials.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as gene product. If the polynucleotide is derived from genomic DNA, expression may include splicing of the mRNA in a eukaryotic cell.

A “subject” as used herein refers to a biological entity containing expressed genetic materials. The biological entity is preferably plant, animal, or microorganisms including bacteria, viruses, fungi, and protozoa. Tissues, cells and their progeny of a biological entity obtained in vivo or cultured in vitro are also encompassed.

A “vector” is a nucleic acid molecule, preferably self-replicating, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription arid/or translation of the DNA or RNA. Also included are vectors that provide more than one of the above functions.

An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

As used herein, the term “compatible vectors” refers to vectors containing the requisite site-specific recombination sites which mediate the recombination of sequences flanked thereby. Thus, two vectors are considered compatible if they contain the compatible site-specific recombination sequences which allow recombination of the flanked sequences.

A “replicon” refers to a polynucleotide comprising an origin of replication (generally referred to as an ori sequence) which allows for replication of the polynucleotide in an appropriate host cell. Examples of replicons include episomes (such as plasmids), as well as chromosomes (such as the nuclear or mitochondrial chromosomes).

Construction of Vectors Encoding Single-Chain Antigen-Binding Units (Sc Abus) of the Present Invention

A central aspect of the present invention is the design of a vector suited for generating Abus in both prokaryotic and eukaryotic cells. The invention vectors are particularly useful for generating a genetically diverse repertoire of Abus, either Sc Abus or Nsc Abus, to facilitate an in vivo screening of Abus that bind to a desired antigen inside a cell. Distinguished from the previously described phagemid vectors (U.S. Pat. No. 5,733,743) and the yeast expression vectors (WO 005/54057), the subject vectors have the following unique characteristics: (a) the vectors replicate and direct expression of Sc Abus in both prokaryotic and eukaryotic cells; and (b) the vectors comprise site-specific recombination sequences that yield a diverse repertoire of Sc Abus in the presence of suitable recombinase, thus facilitating screening of Sc Abus with desired intracellular binding capabilities. In addition, the vectors can be packaged as phage particles in prokaryotic cells upon addition of helper phages. The subject Sc Abus encoding vectors may be further distinguished from the previously employed vectors at the structural level as detailed below.

In one embodiment, the present invention provides a vector replicable in both prokaryotic and eukaryotic cells. The vector comprises a polynucleotide encoding a single-chain antigen-binding unit. The polynucleotide comprises: (a) a variable region of a first antibody chain; (b) a first site-specific recombination sequence; (c) a variable region of a second antibody chain; and (d) a second site-specific recombination sequence; wherein the two site-specific recombination sequences facilitate recombination of the variable regions of (a) and (c) between two compatible vectors.

In another embodiment, the present invention provides a vector replicable in both prokaryotic and eukaryotic cell. The vector comprises a polynucleotide encoding a single-chain antigen-binding unit fused to a gene activation moiety. The polynucleotide comprises: (a) a variable region of a first antibody chain; (b) a first site-specific recombination sequence; (c) a variable region of a second antibody chain fused to a gene activation moiety region; and (d) a second site-specific recombination sequence; wherein the two site-specific recombination sequences facilitate recombination of the variable regions of (a) and (c) between two compatible vectors, and wherein the gene activation moiety facilitates detection of specific binding to an antigen in a eukaryotic cell.

Several factors apply to the design of vectors having the above-mentioned characteristics. First, the vector comprises at least two origins of replication. At least one first origin facilitates replication of the vector in a prokaryotic cell, and at least the one second origin facilitates replication of the vector in a eukaryotic cell. Preferred prokaryotic replicons are replicons capable of directing vector replication in bacterial cells. Non-limiting examples of this class of replicons include pMB1 and pUC. Representative replicons suitable for replicating a vector in eukaryotic cells include the yeast 2u replicon, and a variety of viral replicons including sequences derived from DNA viruses such as Simian Viruses, Geminivirus, Caulimoviridae, Badnaviridae; Circoviridae, Circinoviridae, Parvoviridae, Papovaviridae, Polyomaviridae, Adenoviridae, Herpesviridae, Poxviridae, Iridoviridae, Baculoviridae, Hepadnaviridae, Gyrovirus, Nanovirus, and African Swine Fever virus, or the like.

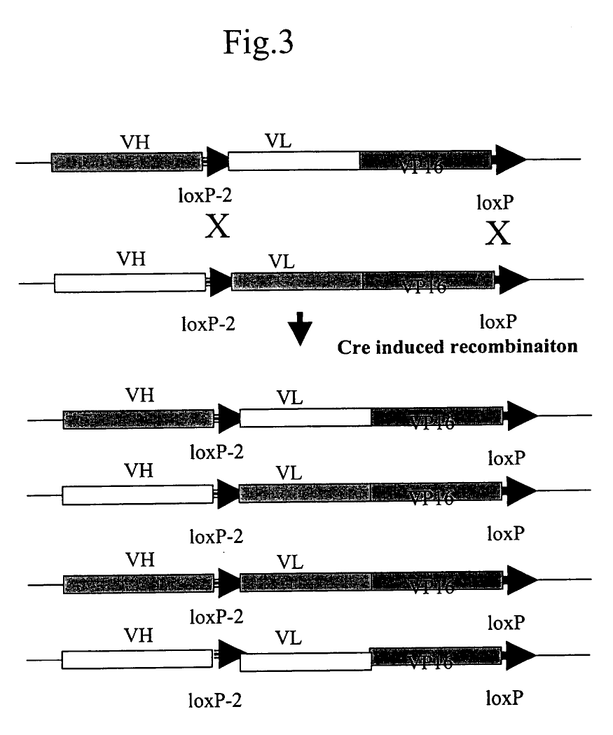

A second consideration in designing the subject vector is to select two site-specific recombination sequences. Recombination is a process whereby genetic exchange occurs between polynucleotide segments. Site specific recombination refers to the process where recombination or shuffling of polynucleotide segments occurs between specific sequences. Such a sequence-specific recombination is typically carried out by site-specific recombinases at two “site-specific recombination sequences,” which in turn dictate the recombination of polynucleotide segments flanked by the two sequences. Preferably, the two site-specific recombination sequences are arranged to flank a variable region of an antibody chain, either the VL or VH region, to effect shuffling of the variable regions and thus generating a diverse repertoire of Sc Abus. More preferably, the two site-specific recombination sequences are distinct sequences (see FIG. 3) to avoid intra-molecular recombination, which may result in gene segment deletion. The shuffling events may take place between two prokaryotic vectors or two eukaryotic vectors, or between a prokaryotic and a eukaryotic vector. In particular, the inclusion of the site-specific recombination sites conveniently effects the transfer of the whole or part of the Sc Abu sequence from one vector to another vector without subcloning the whole or part of the Sc Abu sequence. Such application is particularly advantageous in testing the intracellular binding capabilities of a plurality of Sc Abus when their in vitro binding capabilities have previously been established. For instance, the whole or part of the Sc Abus isolated by conventional phage display technology can be readily shuffled into a yeast vector of the present invention if the Sc Abu sequences are flanked by two site-specific recombination sequences. Similarly, Sc Abus exhibiting the desired intracellular binding capabilities (e.g. as determined by the two-hybrid systems detailed below) can be readily shuffled from the subject yeast vector into an animal cell vector that also contains the corresponding site-specific recombination sequences. The ability to efficiently transfer the Sc Abus greatly facilitates the generation and expression of a genetically diverse repertoire of Sc Abus in a variety of vectors without involving laborious subcloning steps. As noted above, such an experimental design is particularly important in elucidation of the biological functions of the antigens to which the Abus bind intracellularly.

A preferred site-specific recombination system is the lox P/Cre recombinase system of coliphage P1 (Hoess, R. H. and Abremski, K. (1990) Nucleic acids and Molecular Biology). Cre-recombinase catalyses a highly specific recombination event at sequences called lox. For instance, loxP, the recombination site in phage P1 consists of two 13 bp inverted repeats separated by an 8 bp non-symmetrical core. The recombination is highly efficient, and sequence-specific for loxP site, which can be readily incorporated into the vectors of the present invention. As used herein, the term “loxP sequence” encompasses the wildtype loxP sequence, loxP derivatives or mutants. The derivatives and mutants comprise sequences that are derived from the wildtype loxP sequences. Preferred loxP derivatives or mutants mediate recombination among mutant loxP sites and not with the wildtype loxP sequences. Preferred loxP derivatives or mutants include but are not limited to loxP2, and loxP511.

Another site-specific recombination system suitable for constructing the subject vectors is the Frt/Flp recombinase system. Flp recombinase catalyzes a site-specific recombination reaction that is involved in amplification of the 2u plasmid of S. cerevisiare (Cox et al. (1983) PNAS 80:4223-4227. Frt is the Flp target sequences, and analogous to the loxP site, it has two 13 base-pair repeats, separated by an 8 base-pair spacer sequence. The target sequence is as follows: GAAGTTCCTATTCTCTAGAAAGTATAGGAACTTC. As used here, the term “Frt sequence” encompasses the wildtype Frt sequence, Frt derivatives or mutants. The derivatives and mutants comprise sequences that are derived from the wildtype Frt sequences.

Other well-characterized site-specific recombination system are the ones used in integration and excision of bacteriophage lambda (In “Echerichia coli and Salmonella typhimurium. Cellular and Molecular Biology.” (1987), pp 1054-1060. Neidhart, F. C. Editor in Chief. American Society for Microbiology). This bacteriophage can follow two developmental pathways once inside the cell: lysis or lysogeny. The lysogenic pathway involves integration of the lambda genome into the chromosome of the infected bacterium; integration is the result of a site-specific recombination between a ca. 240 bp sequence in the bacteriophage called art P and a 25 bp site in the bacterial chromosone called art B. The integration event is catalysed by a host-encoded factor called IHF and a phage encoded enzyme called Int recombinase, which recognizes a 15 bp region common to the two att sites. The integrated DNA is flanked by sequences derived from art B and art P, and these are called att L and att R. The integration event is reversible and is catalysed by Int, IHF and a second bacteriophage encoded enzyme, Xis. This system can readily be modified to transfer segments between replicons within E. coli. For example, the donor gene could be flanked by att L and att R sites such that when Int and Xis proteins are provided in the host cell, recombination between att L and att R sites would create a circular DNA segment containing the donor gene and a recreated att B site. This circular segment could then recombine with an att P site engineered into the recipient plasmid.

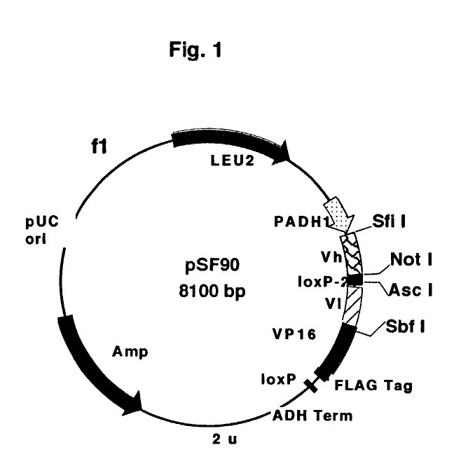

In Example 1 and FIG. 1, a VH and a VL region are cloned into a vector containing a phage replication origin (f1 ori), a bacterial origin (pUC ori) and a yeast replication origin (2u). The two variable regions are linked together via a loxP2 site. Another loxP site is placed downstream of the single-chain polypeptide coding regin. The VL region is fused with the VP 16 transcription activation domain. This construct allows efficient recombination of VH and VL regions from a low complexity Sc Abus library to generate a vast diverse repertoire of Sc Abus that can be readily screened in a two-hybrid system.

In constructing the subject vectors, the polynucleotide sequences corresponding to various regions of L or H chain of an existing antibody can be readily obtained and sequenced using conventional techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection (http://www.atcc.org), which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al.(1989) Proc. Natl. Acad. Sci. U.S.A. 86: 3833-3837; Larrick et al. (1989) Biochem. Biophys. Res. Commun. 160:1250-1255; Sastry et al. (1989) Proc. Natl. Acad. Sci., USA. 86: 5728-5732; and U.S. Pat. No. 5,969,108.

The antibody nucleotide sequences may also be modified, for example, by substituting the coding sequence for human heavy and light chain constant regions in place of the homologous non-human sequences. In that manner, chimeric antibodies are prepared that retain the binding specificity of the original antibody.

The antibody nucleotide sequences may also be derived from synthetic oligonucleotide sequences that are inserted in one or more CDR regions in the VH or VL regions.

The polynucleotides embodied in the invention include those coding for functional equivalents and fragments thereof of the exemplified polypeptides. Functionally equivalent polypeptides include those that enhance, decrease or do not significantly affect properties of the polypeptides encoded thereby. Functional equivalents may be polypeptides having conservative amino acid substitutions, analogs including fusions, and mutants.

Due to the degeneracy of the genetic code, there can be considerable variation in nucleotides of the L and H sequences suitable for construction of the polynucleotides and vectors of the present invention. Sequence variants may have modified DNA or amino acid sequences, one or more substitutions, deletions, or additions, the net effect of which is to retain the desired antigen-binding activity. For instance, various substitutions can be made in the coding region that either do not alter the amino acids encoded or result in conservative changes. These substitutions are encompassed by the present invention. Conservative amino acid substitutions include substitutions within the following groups: glycine, alanine; valine, isoleucine, leucine; aspatic acid, glutamic acid; asparagine, glutamine; serine, threonine; lysine, arginine; and phenylalanine, tyrosine. While conservative substitutions do effectively change one or more amino acid residues contained in the polypeptide to be produced, the substitutions are not expected to interfere with the antigen-binding activity of the resulting Abus to be produced. Nucleotide substitutions that do not alter the amino acid residues encoded are useful for optimizing gene expression in different systems. Suitable substitutions are known to those of skill in the art and are made, for instance, to reflect preferred codon usage in the expression systems.

Where desired, the recombinant polynucleotides may comprise heterologous sequences that facilitate detection of the expression and purification of the gene product. Examples of such sequences are known in the art and include those encoding reporter proteins such as β-galactosidase, β-lactamase, chloramphenicol acetyltransferase (CAT), luciferase, green fluorescent protein (GFP) and their derivatives. Other heterologous sequences that facilitate purification may code for epitopes such as Myc, HA (derived from influenza virus hemagglutinin), His-6, FLAG, or the Fc portion of immunoglobulin, glutathione S-transferase (GST), and maltose-binding protein (MBP).

The polynucleotides can be conjugated to a variety of chemically functional moieties described above. Commonly employed moieties include labels capable of producing a detectable signal, signal peptides, agents that enhance immunologic reactivity, agents that facilitate coupling to a solid support, vaccine carriers, bioresponse modifiers, paramagnetic labels and drugs. The moieties can be covalently fused polynucleotide recombinantly or by other means known in the art.

The polynucleotides of the invention can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

The polynucleotides embodied in this invention can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

In certain preferred embodiment, the encoded Sc Abu is expressed as a fusion with a gene activation moiety. The gene activation moiety facilitates the detection of specific binding of the Sc Abu to an antigen in a eukaryotic cell. Such a specific binding is preferably detected in a yeast cell employing a two-hybrid system.

The yeast two-hybrid system and its derivative systems have been widely used to detect protein-protein interactions (see, e.g. U.S. Pat. Nos. 5,283,173, 5,965,368, 5,948,620, 6,171,795, 6,132,963, 5,695,941, 6,187,535, 6,159,705, 6,057,101, 6,083,693, 5,928,868, 6,200,759, WO 95/14319, WO 95/26400). These well-established systems generally involve in vivo reconstitution of two separable domains of a transcription factor. The DNA-binding domain (DB) of the transcription factor is required for recognition of a chosen promoter. The transcription activation domain (AD) is required for contacting other components of the cell's transcriptional machinery. In these systems, the transcription factor is reconstituted through the use of hybrid proteins. One hybrid is composed of the AD and a first protein of interest. The second hybrid is composed of the DB and a second protein of interest. In detecting specific binding of an Abu to a desired antigen, the Abu is typically fused with the AD and the antigen is fused to the DB domain. Alternatively, the Abu is fused with the DB, and the antigen is fused to the AD. In case where the Abu binds to the antigen of interest, the AD and DB are brought into close physical proximity, thereby reconstituting the transcription factor. Specific binding of an Abu to a desired antigen can be measured by assaying the ability of the reconstituted transcription factor to activate transcription of a reporter gene.

The term “DNA-binding domain” or “DB” means a polypeptide sequence that is capable of directing specific polypeptide binding to a particular DNA sequence (i.e., to a DNA-binding-protein recognition site or “DNA-BPRS). The term “domain” in this context is not intended to be limited to a discrete folding domain. Rather, consideration of a polypeptide as a DB for use in the fusion protein can be made simply by the observation that the polypeptide has a specific DNA-binding activity. Non-liminting examples of DB containing proteins are GAL4, LexA, and ACE1. As is apparent to one of ordinary skill in the art, the DNA binding domain need not be derived from proteins in a prokaryotic cell. Proteins of eukaryotic origin and exhibiting desired DNA binding activity can be used. For example, the DB portion of the fusion protein can include polypeptide sequences from eukaryotic DNA binding proteins as p53, Jun, Fos, GCN4, or GAL4. Likewise, the DNA binding portion of the fusion protein can be generated from viral proteins, such as the pappillomavirus E2 protein. Alternatively, the DNA binding domain can be generated by combinatorial mutagenic techniques, and represent a DB not naturally occurring in any organism. A variety of techniques have been described in the art for generating novel DNA binding proteins which can selectively bind to a specific DNA sequence (see, e.g. U.S. Pat. No. 5,198,346).

Where desired, the DNA binding domain can include oligomerization motifs. It is well known in the art that certain transcriptional regulators dimerize, with dimerization promoting cooperative binding of the two monomers to their cognate recognition elements. For example, where the fusion protein includes a LexA DNA binding domain, it can further include a LexA dimerization domain. This optional domain facilitates efficient LexA dimer formation. Because LexA binds its DNA binding site as a dimer, inclusion of this domain in the bait protein also optimizes the efficiency of operator occupancy (Golemis and Brent, (1992) Mol. Cell Biol. 12:3006). Other oligomerization motifs useful in the present invention will be readily recognized by those skilled in the art. Exemplary motifs include the tetramerization domain of p53 and the tetramerization domain of BCR-ABL. In addition, a variety of techniques are known in the art for identifying other naturally occurring oligomerization domains, as well as oligomerization domains derived from mutant or otherwise artificial sequences. See, for example, Zeng et al. (1997) Gene 185:245.

The term “gene activation moiety” refers to a stretch of amino acids capable of inducing the expression of a gene whose control region (i.e. the promoter) is bound. A variety of gene activation moieties containing transcription activation domains are available in the art for constructing the subject vectors. Generally, the transcription activation domain of any transcription factor can be used. A preferred example is VP16. All of the essential elements of a two-hybrid system, which include the DNA-binding-protein recognition site, the transcription activation, and the DNA-binding domain, may correspond to one transcription factor, or they can correspond to different transcription factors. Suitable DNA-binding-protein recognition sites include those for the yeast protein GAL4, the bacterial protein LexA, the yeast metal-binding factor Ace1. These binding sites can readily be used with a repressed promoter (e.g., a SPO13 promoter can be used as the basis for SPAL, SPEX and SPACE promoters, respectively, for a SPO13 promoter combined with GAL4, LexA, and ACE1 DNA binding sites). Other useful transcription factors include the GCN4 protein of S. cerevisiae (see, e.g., Hope and Struhol, 1986, Cell 46:885-894) and the ADR1 protein of S. cerevisiae (see, e.g., Kumar et al., 1987, Cell 51:941-951).

The term “reporter gene” means a gene whose expression can be assayed as a measure of the ability of an Abu to bind to an antigen of particular interest. The reporter genes may encode any protein that provides a phenotypic marker, for example: a protein that is necessary for cell growth or a toxic protein leading to cell death, e.g., a protein which confers antibiotic resistance or complements an auxotrophic phenotype; a protein detectable by a colorimetric/fluorometric assay leading to the presence or absence of color/fluorescence; or a protein providing a surface antigen for which specific antibodies/ligands are available. Non-limiting examples of reporter genes are lacZ, amino acid biosynthetic genes (e.g., the yeast LEU2, HIS3, LYS2, or TRP1), URA3 genes, nucleic acid biosynthetic genes, the bacterial chloramphenicol transacetylase (cat) gene, MEL, and the bacterial gus gene. Also included are those genes that encode fluorescent markers, such as the Green Fluorescent Protein gene.

The reporter genes may be further classified as “selectable,” “counterselectable,” or “selectable/counterselectable” reporter genes. By “selectable” reporter gene is meant a reporter gene which, when it is expressed under a certain set of conditions, confers a growth advantage on cells containing it. By “counterselectable” reporter gene is meant a reporter gene which, when it is expressed under a certain set of conditions, inhibits the growth of a cell containing it. Examples of counterselectable reporter genes include well-established marker sequences such as URA3, LYS2, LYS5, GAL1, CYH2, and CAN1. The term “selectable/counterselectable” as applied to a reporter gene refers to the reporter that is lethal to a cell when it is expressed under a certain set of conditions, but confers a selective growth advantage on cells when it is expressed under a different set of conditions. Thus, a single gene can be used as both a selectable reporter gene and a counterselectable reporter gene. Examples of selectable/counterselectable reporter genes include URA3, LYS2, and GAL1. In each aspect of the invention where a selectable/counterselectable reporter gene is employed, a combination of a selectable reporter gene and a counterselectable reporter gene can be used in lieu of a single selectable/counterselectable reporter gene. The reporter genes can be located on a plasmid or can be integrated into the genome of a haploid or diploid cell. Generally, the reporter genes are operably fused to a promoter that is specifically recognized by the DB. The reporter gene whose expression is to be assayed is operably fused to a promoter that has sequences that direct transcription of the reporter gene. The reporter gene is positioned such that it is expressed when a gene activating moiety of a transcription factor is brought into close proximity to the gene (e.g., by using hybrid proteins to reconstitute a transcription factor, or by covalently bonding the gene-activating moiety to a DNA-binding protein). The reporter gene can also be operably fused to regulatory sequences that render it highly responsive to the presence or absence of a transcription factor. For example, in the absence of a specific transcription factor, a highly responsive URA3 allele confers a Ura⁻ Foa^{r} phenotype on the cell. In the presence of a specific transcription factor, a highly responsive URA3 allele confers a Ura⁺ Foa^{s} phenotype on the cell. Where the cell carrying the reporter gene (i.e., a transformed yeast cell) normally contains a wild-type copy of the gene (e.g., the URA3 gene), the exogenous reporter gene can be integrated into the genome and replace the wild-type gene. Conventional methods and criteria can be used to connect a reporter gene to a promoter and to introduce the reporter gene into a cell.

Suitable promoters for expression of a reporter gene are those which, when fused to the reporter gene, can direct transcription of it in the presence of appropriate molecules (i.e., proteins having transcriptional activation domains), and which, in the absence of a transcriptional activation domain, do not direct transcription of the reporter gene. Non-limiting examples of useful promoter are the yeast SPO13 promoter and the pADH1 promoter. Other useful promoters include those promoters which contain upstream repressing sequences (see, e.g., Vidal et al., 1995, Proc. Natl. Acad. Sci. U.S.A. 92:2370-2374) and which inhibit expression of the reporter gene in the absence of a transcriptional activation domain. The ability of a promoter to direct transcription of a reporter gene can be measured with conventional methods of assaying for gene expression (e.g., detection of the gene product or its mRNA, or detection of cell growth under conditions where expression of the reporter gene is required for growth of a cell).

In addition to the above-described elements, the vectors may contain termination sequences. The termination sequences associated with the coding region are typically inserted into the 3′ end of the coding region desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various polyadenylation sequences that are known in the art, widely available, and exemplified herein. Where the terminator comprises a gene, it can be advantageous to use a gene which encodes a detectable or selectable marker; thereby providing a means by which the presence and/or absence of the terminator sequence (and therefore the corresponding inactivation and/or activation of the transcription unit) can be detected and/or selected.

The vectors embodied in this invention can be obtained using recombinant cloning methods and/or by chemical synthesis. A vast number of recombinant cloning techniques such as PCR, restriction endonuclease digestion and ligation are well known in the art, and need not be described in detail herein. One of skill in the art can also use the sequence data provided herein or that in the public or proprietary databases to obtain a desired vector by any synthetic means available in the art.

Host Cells Comprising the Subject Vectors:

The invention provides host cells comprising or transfected with the vectors or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For prokaryotes and eukaryotic microbes such as fungi or yeast cells, any of the above-mentioned methods is suitable for vector delivery. Suitable prokaryotes for this purpose include bacteria including Gram-negative and Gram-positive organisms. Representative members of this class of microorganisms are Enterobacteriaceae (e.g E. coli), Enterobacter, Erwinia, Klebsiella, Proteus, Salmonella (e.g. Salmonella typhimurium), Serratia (e.g., Serratia marcescans), Shigella, Neisseria (e.g. Neisseria meningitidis) as well as Bacilli (e.g. Bacilli subtilis and Bacilli licheniformis). Preferably, the host cell secretes minimal amounts of proteolytic fragments of the expressed Abus. Commonly employed fungi (including yeast) host cells are S. cerevisiae, Kluyveromyces lactis (K. lactis), species of Candida including C. albicans and C. glabrata, C. maltosa, C. utilis, C. stellatoidea, C. parapsilosis, C. tropicalus, Neurospora crassas, Aspergillus nidulans, Schizosaccharomyces pombe (S. pombe), Pichia pastoris, and Yarowia lipolytica.

To perform the two-hybrid screening method, the suitable yeast strains can be grown and maintained according to standard methods. Saccharomyces cerevisiae are particularly useful in the invention. In certain aspects of the invention, mating of two mating competent yeast cells is desired. For example, in certain methods, a hybrid protein that includes an activation domain is expressed in one mating competent cell, and a hybrid protein that includes a DNA-binding domain is expressed in a second mating competent cell. In such a case, the transcription factor is reconstituted by mating the first and second mating competent cells. As is apparent to artisans in the field, the two mating competent cells should be of compatible mating types. For example, one mating competent cell can be of the MATa mating type, and the other mating competent cell can be of the MATα mating type. It is inconsequential which hybrid protein is expressed in which cell type. A preferred yeast cell for screening Abus that is immunoreactive with a desired antigen contains a counterselectable reporter gene which is operably fused to a promoter which facilitates elimination of yeast cells expressing the counterselectable reporters independent of the specific binding of a test Abu to an antigen of interest. In addition, a yeast cell can contain, integrated into its genome, a selectable marker (e.g., HIS3) and/or a gene whose expression can be screened (e.g., lacZ).

The above-mentioned delivery methods are also suitable for introducing vectors to most of the animal cells. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

The animal cells can be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, animal cells can be grown in a defined medium that lacks serum but is supplemented with hormones, growth factors or any other factors necessary for the survival and/or growth of a particular cell type. Whereas a defined medium supporting cell survival maintains the viability, morphology, capacity to metabolize and potentially, capacity of the cell to differentiate, a defined medium promoting cell growth provides all chemicals necessary for cell proliferation or multiplication. The general parameters governing mammalian cell survival and growth in vitro are well established in the art. Physicochemical parameters which may be controlled in different cell culture systems are, e.g., pH, pO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, most cells also require one or more hormones from at least one of the following groups: steroids, prostaglandins, growth factors, pituitary hormones, and peptide hormones to proliferate in serum-free media (Sato, G. H., et al. in “Growth of Cells in Hormonally Defined Media,” Cold Spring Harbor Press, N.Y., 1982). In addition to hormones, cells may require transport proteins such as transferrin (plasma iron transport protein), ceruloplasmin (a copper transport protein), and high-density lipoprotein (a lipid carrier) for survival and growth in vitro. The set of optimal hormones or transport proteins will vary for each cell type. Most of these hormones or transport proteins have been added exogenously or, in a rare case, a mutant cell line has been found which does not require a particular factor. Those skilled in the art will know of other factors required for maintaining a cell culture without undue experimentation.

Once introduced into a suitable host cell, expression of the Abus can be determined using any nucleic acid or protein assay known in the art. For example, the presence of transcribed mRNA of L or H chain, or the Sc Abu can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of Abu polynucleotide.

Expression of the vector can also be determined by examining the Abu expressed. A variety of techniques are available in the art for protein analysis. They include but are not limited to radioimmunoassays, ELISA (enzyme fused immunoradiometric assays), “sandwich” immunoassays, immunoradiometric assays, in situ immunoassays (using e.g., colloidal gold, enzyme or radioisotope labels), western blot analysis, immunoprecipitation assays, immunoflourescent assays, and PAGE-SDS.

Uses of the Polynucleotides, Vectors and Host Cells of the Present Invention:

The polynucleotides and vectors of this invention have several specific uses. They are useful, for example, in expression systems for the production of the Sc Abus. The polynucleotides are useful as primers to effect amplification of desired polynucleotides. Furthermore, The polynucleotides of this invention are also useful in pharmaceutical compositions including vaccines, diagnostics, and drugs.

The host cells of this invention can be used, inter alia, as repositories of the subject polynucleotides, vectors, or as vehicles for producing and screening desired Abus based on their antigen binding specificities.

Accordingly, the invention provides a method of generating a selectable library of vectors that encodes a genetically diverse repertoire of Sc Abus. The method is particularly useful for producing an extremely diverse repertoire of Sc Abus that is amenable to selection in a two-hybrid system. The method involves the following steps: (a) providing a plurality of vectors of the subject vectors that contain the gene-activation moiety; (b) causing or allowing site-specific recombination of the variable regions encoded by at least two compatible vectors, thereby generating the selectable library. In one aspect, the recombination may occur in vitro in the presence of a site-specific recombinase. Preferably, the site-specific recombinase is in soluble form. In another aspect, the recombination may take place in a cell that expresses a site-specific recombinase. The recombinase may be expressed by a vector contained in the cell, or as an integral part of the genome of the cell. In case of in vivo recombination, the step of providing a plurality of the subject vectors further involves the steps of: (a) introducing a plurality of the vectors into a population of prokaryotic cells; (b) infecting a first population of prokaryotic cells with a plurality of helper phages to yield a population of phage particles; and (c) infecting a second population of prokaryotic cells with the phage particles of (b); and optionally repeating the step of (c), thereby introducing a plurality of the vectors into a cell. Distinguished from the conventional process of plasmid transfection, which typically delivers one copy into a bacterial cell, this instant method involves phage particles that are capable of performing “multiplicity of infection,” thus delivering a plurality of the subject vectors into a host cell, in which site-specific recombination can take place. As described herein, a variety of site-specific recombination systems available in the art can be employed in the subject method of producing a repertoire of Sc Abus. A preferred system is the loxP/Cre-recombinase system described above.

Following the recombination, a vastly diverse repertoire of Sc Abus, each being fused to a gene activation moiety is generated. The recombined repertoire has a complexity ranging from about 10⁶ to about 10¹³, and preferably from about 10⁷ to about 10⁹. A more preferred range is from about 10⁸ to about 10¹⁰, and more preferably from about 10⁸ to about 10¹¹. Even more preferred is a range from about 10⁹ to about 10¹⁰, and yet even more preferably from about 10⁹ to about 10¹¹.

The gene activation moiety fused with the Sc Abus enables the detection of specific binding of the Sc Abus and a desired antigen inside a cell. The preferred detection system is the two-hybrid system and improvements thereof. Methods and procedures to perform yeast two-hybrid screening are well-established in the art and thus are not detailed herein. Upon detecting a specific binding, the nucleic acid encoding the Sc Abu that exhibits the desired intracellular binding capability can readily be isolated by any conventional recombinant DNA techniques.

Where desired, the repertoire of Sc Abus can be pre-selected against an unrelated antigen to counter-select the undesired Abus. The repertoire may also be pre-selected against a related antigen in order to isolate, for example, anti-idiotypic Abus.

The subject Sc Abu repertoire enables rapid isolation of Sc Abus with desired specificities. Many of the isolated Sc Abus would be expected to be difficult or impossible to obtain through conventional hybridoma or transgenic animal technology. In addition, these antigen-binding units capable of binding to their respective antigens (i.e. “intracellular” antigen-binding units) inside a cell are of tremendous research and therapeutic value. The ability of these binding units to specifically inhibit a protein's function and/or expression allows one to elucidate the biological function of the protein by creating essentially a protein-specific “knock-out” cell. Thus, the generation of these antibodies greatly facilitates functional genomics studies.

Kits Comprising the Vectors of the Present Invention

The present invention also encompasses kits containing the vectors of this invention in suitable packaging. Kits embodied by this invention include those that allow generation of Sc Abus that are fused to gene activation moieties.

Each kit necessarily comprises the reagents that render the delivery of vectors into a host cell possible. The selection of reagents that facilitate delivery of the vectors may vary depending on the particular transfection or infection method used. The kits may also contain reagents useful for generating labeled polynucleotide probes or proteinaceous probes for detection of Abus. Each reagent can be supplied in a solid form or dissolved/suspended in a liquid buffer suitable for inventory storage, and later for exchange or addition into the reaction medium when the experiment is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information.

Further illustration of the development and use of Sc Abus vector libraries, polynucleotides, vectors and host cells according to this invention are provided in the Example section below. The examples are provided as a guide to a practitioner of ordinary skill in the art, and are not meant to be limiting in any way.

### EXAMPLES

### Example 1

### Construction of Vectors Encoding Single-Chain Antigen-Binding Units Fused to Gene Activation Moieties

A variety of vectors having the unique features as described above can be generated using conventional recombinant DNA techniques. By way of illustration, we have constructed a phagemid vector pSF90 (FIG. 1) that expresses single chain VH-VL fused by loxP-2 site with c-terminal fusion to the transcription activation domain VP 16. pSF90 was constructed as follows. First, the VP16 transcriptional activation domain was synthesized using oligos and PCR assembly techniques known in the art. The NLS (nuclear localization sequence) was added at the N-terminal, and the FLAG tag was added at the C-terminal of the VP16 activation domain. The gene fragment was cloned into the two Hind III sites of pGADT7 vector and thus replacing the Hind III fragment containing the Gal4 AD in the pGADT7 vector. The anti-ras antibody Y238 (Cochet et al., (1998) Molecular Immunology. 35:1097-1110) was synthesized using oligos and PCR techniques known in the fields, the assembled Y238 anti-ras antibody heavy chain was attached with Sfi I and Not I restriction sites at the N-terminal and C-terminal sequence respectively, and the assembled Y238 anti-ras antibody light chain was attached with Asc I and Sbf I restriction sites at the N-terminal and C-terminal sequence respectively. The two fragments were linked with loxP2 site. A second loxP site with wild-type sequence was incorporated downstream of the VP16 coding sequences. This plasmid has amp marker for selection in E. coli, and Leu2 for selection in yeast.

In addition, the vector also carries a fl origin. The fl ori carries the sequences required in cis for initiation and termination of bacteriophage particles. When cells harboring these plasmids are infected with a helper phage such as M13K07, progeny of phage particles containing the cloned or library genetic information are generated, which in turn are infectious to suitable host E. coli strains. Furthermore, phage particles, unlike the plasmid transformation, can infect one cell with multiple phage particles, as can be determined by the multiplicity of infection (M.O.I).

### Example 2

### Preparation of Host Cells Comprising Vectors of the Present Invention

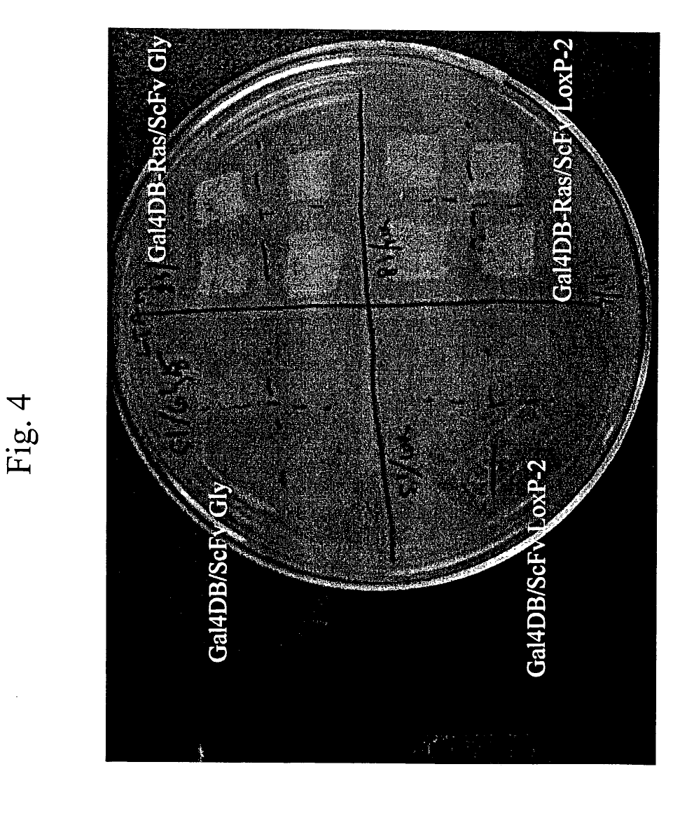

Host cells transformed with the invention vectors can be prepared using any known procedures in the art and/or any methods described herein. We have prepared yeast cells carrying the above-described expression vector. The plasmid pSF90 that expresses the single chain VH-loxP2-VL-VP16-flag fusion protein was transformed into the yeast strain AH109. The plasmid library expressing the Ras antigen was transformed into the yeast strain Y187. Mating of these two strains of cells was carried out according to the well-established procedures in the art (see, e.g. Methods in Enzymology (Academic Press, San Diego) 194:1-932). After mating, the diploid cells expressing the single chain VH-loxP2-VL-VP16-flag fusion protein that exhibits intracellular binding affinity to the RAS antigen were selected based on their ability to grown in selective media (FIG. 4).

Where desired, the vectors encoding the antigen and Sc Abus with the desired binding affinity can be isolated from the selected yeast cells. The isolated vectors can then be used to transform E. coli strain for storage or for further amplification. Specifically, Ampicillin containing plates were used to select and propagate vectors encoding the single-chain fusion protein. Kanamycin containing plates were used to select and propagate vectors encoding the antigen.

### Example 3

### Construction of Genetically Diverse Repertoire of Single-Chain Antigen-Binding Units Suitable for In vivo Screening

a) PCR Amplification of VH and VL and Construction of VH-loxP2-VL-VP16 Hybrid Expression Library:

To optimize the coverage of the diversity of the antibody genes, we take the advantage of the recent completion of human genome sequence and the catalogue of all the functional germline V genes in the database. The design of the primer pairs therefore are aimed at recognizing all the genes, or as many as possible. First, the V gene encoding the CDR1 and CDR2 from both germline or rearranged mRNA are PCR amplified using primers corresponding to the N-terminal of the domains, and the frame 3 regions of both heavy and light chain. Next, the CDR3 is amplified using the primers corresponding to the frame 3 and the J segments of both heavy and light chain. As VJ in light chain or VDJ in heavy chain DNA rearrangement in lymphocytes, the first PCR product and the second PCR product is combined through recombinant PCR, with addition of the restriction site Sfi I on the N-terminal and Not I at the C-terminal for the heavy chain, and of the restriction site Asc I and Sbf I for the light chain. In this way, each V gene is recombind randomly with the CDR3 and thus increases the complexity of the repertoire. The recombinatorial VH library is then digested with Sfi I and Not I, and ligated to the vector pSF90, yielding a library of VH. The recominatorial VL library is cut with Asc I and Sbf I, and ligated to above said VH library in vector pSF90, cut with Asc I and Sbf I, resulting libraries of VH-loxP2-VL-VP16 fusion protein.

b) VH/VL Diversification Through Cre-Induced Recombination at loxP and loxP-2 Sites.

The single chain library constructed as described above in vector pSF90 are transformed into E. coli, and then infected with helper phage M13KO7, and phage particles are isolated. The phages are used to infect E. coli at multiplicity of infection 20:1, P1 phages are used to infect the host E. coli to express the Cre recombinase so that the recombination between the wild-type loxP sites and between the mutant loxp-2 sites among different clones of the libraries can occur, resulting in the shuffling of the VL-Vp16-flag domain among different clones. Plasmid DNA are then isolated from E. coli and transformed into yeast AH109, and mated with Y187 expressing the desired antigen or a cDNA library fused with the Gal4 DNA binding (DB) domain. The mating can be carried out as described in the field (Guthrie, C & Fink G. R. 1991. Guide to Yeast Genetics and Molecular Biology. In Methods in Enzymology (Academic Press, San Diego) 194:1-932). After mating, the diploid cells are subject to selection on selective media, selecting for growth of cells expressing the single chain VH-loxP2-VL-VP16-flag fusion protein that specifically recognize the expressed antigen protein from cDNA libraries or a desired specific antigen in synthetic selection media.

c) Recovery of Antigen and Ab Expression Plasmid in E. coli

The DNA is prepared and isolated from yeast as described (Guthrie, C & Fink G. R. 1991. Guide to Yeast Genetics and Molecular Biology. In Methods in Enzymology (Academic Press, San Diego) 194:1-932), and transformed into E. coli strain, and the transformation is plated on different selection plates, on Amp plate for the plasmid expressing the single chain VH-loxP2-VL-VP16-flag fusion protein, and kan plate for the plasmid expressing antigen. The plasmid DNA is subject to sequence analysis. DNA sequence analysis can be used to determine the identity of the antigen and the antigen binding fragments.

### Example 4

### Construction of Host Strain that Counterselect Non-Specific Antigen-Binding Units

A host strain capable of counterselecting non-specific Abus can be generated as follows. It has been previously characterized that the cyh2 gene encodes the L29 ribosome subunit. Cycloheximide blocks polypeptide elongation during translation and prevents cell growth. However, a cycloheximide resistance allele cyh2r was identified (Kaufer et al. (1983) Nucleic Acids Res. 11:3123) due to a single amino acid change in the cyh2 protein. The sensitivity of the wild type cyh2 protein to the drug is dominant and thus the cells expressing both the wild-type and mutant cyh2 protein fail to grow on media containing cycloheximide. In this counter-selection scheme, the endogenous cyh2 gene is replaced with the mutant allele cyh2r. The wild-type cyh2 is introduced as transgene under the control of a LexA binding site (LexA operation sequence). In this same host strain, LexA DNA binding domain is fused with an unrelated antigen, which may be expressed from a chromosome location or plasmid. If the selected antigen-binding unit is non-specific to an antigen of interest (i.e. it also binds to the unrelated antigen), then the VP16 activation domain will be brought to proximity to the LexA binding site and drive the expression of counterselectable reporter cyh2. As a result, cells expressing cyh2 are killed in the presence of cycloheximide, thus facilitating a specific selection of those cells expressing antigen-binding units specifically binding to the desired antigen. Aside from cyh2, SUP4-o and CAN1 can also be used as the counterselectable marker.

What is claimed is:

1 A vector replicable in both a prokaryotic and eukaryotic cell, comprising a polynucleotide encoding a single-chain antigen-binding unit, said polynucleotide comprising:
a) a variable region of a first antibody chain;
b) a first site-specific recombination sequence;
c) a variable region of a second antibody chain; and
d) a second site-specific recombination sequence;
wherein the two site-specific recombination sequences facilitate recombination of the variable regions of (a) and (c) between two compatible vectors.

2. The vector of claim 1, wherein the first antibody chain is light chain and the second antibody chain is heavy chain.

3. The vector of claim 1, wherein the first antibody chain is heavy chain and the second antibody chain is light chain.

4. The vector of claim 1, further comprising at least two origins of replication, wherein at least one first origin facilitates replication in a prokaryotic cell, and at least one second origin facilitates replication in a eukaryotic cell.

5. The vector of claim 1, wherein the first and second site-specific recombination sequences are different sequences.

6. The vector of claim 1, wherein the first site-specific recombination sequences is loxP sequence, and the second site-specific recombination sequences is loxP2.

7. The vector of claim 1, wherein the first site-specific recombination sequences is loxP2 sequence, and the second site-specific recombination sequences is loxP.

8. The vector of claim 1, wherein the first and/or the second site-specific recombination sequence is Frt sequence.

9. The vector of claim 1, wherein the prokaryotic cell is bacterium.

10. The vector of claim 9, wherein the bacterium is E. coli.

11. The vector of claim 1, wherein the eukaryotic cell is a yeast cell.

12. The vector of claim 11, wherein the yeast cell is S. cerevisiae.

13. A host cell comprising a vector of claim 1.

14. A vector replicable in both prokaryotic and eukaryotic cell, comprising a polynucleotide encoding a single-chain antigen-binding unit fused to a gene activation moiety, said polynucleotide comprising:
(a) a variable region of a first antibody chain;
(b) a first site-specific recombination sequence;
(c) a variable region of a second antibody chain fused to a gene activation moiety region; and
(d) a second site-specific recombination sequence;
wherein the two site-specific recombination sequences facilitate recombination of the variable regions of (a) and (c) between two compatible vectors, and wherein the gene activation moiety facilitates detection of specific binding to an antigen in a eukaryotic cell.

15. The vector of claim 14, wherein the detection of specific binding employs a two-hybrid system.

16. The vector of claim 14, further comprising at least two origins of replication, wherein at least one first origin facilitates replication in a prokaryotic cell, and at least one second origin facilitates replication in a eukaryotic cell.

17. The vector of claim 14, wherein the second origin facilitates replication in yeast cell.

18. The vector of claim 14, further comprising at least one gene encoding a selectable marker.

19. The vector of claim 14, wherein the first antibody chain is a heavy chain, and the second antibody chain is a light chain.

20. The vector of claim 14, wherein the first antibody chain is a light chain, and the second antibody chain is a heavy chain.

21. The vector of claim 14, wherein the variable region comprises variable region sequences of a human antibody.

22. The vector of claim 14, wherein the variable region comprises variable region sequences of a non-human antibody.

23. The vector of claim 14, wherein the gene activation moiety comprises a transcription activation domain of a protein selected from the group consisting of GAL4 and VP16.

24. The vector of claim 14, wherein the first and second site-specific recombination sequences are different sequences.

25. The vector of claim 14, wherein the first or the second site-specific recombination sequence is loxP sequence.

26. The vector of claim 14, wherein the first site-specific recombination sequences is loxP sequence, and the second site-specific recombination sequences is loxP2 sequence.

27. The vector of claim 14, wherein the first site-specific recombination sequences is loxP2 sequence, and the second site-specific recombination sequences is loxP sequence.

28. The vector of claim 14, wherein the first and/or the second site-specific recombination sequence is Frt sequence.

29. The vector of claim 14, wherein the prokaryotic cell is bacterium.

30. The vector of claim 29, wherein the bacterium is E. coli.

31. The vector of claim 14, wherein the eukaryotic cell is a yeast cell.

32. The vector of claim 31, wherein the yeast cell is S. cerevisiae.

33. The vector of claim 14, further comprising a promoter 5′ to the variable region of the first antibody chain.

34. A host cell comprising a vector of claim 14.

35. A library of vectors of claim 14, wherein each vector of the library encoding a unique single-chain antigen-binding unit with respect to all other vectors of the library.

36. A method of generating a selectable library of vectors encoding a genetically diverse repertoire of single-chain antigen-binding units, comprising:
(a) providing a plurality of vectors of claim 14;
(b) causing or allowing site-specific recombination of the variable regions (a) and (c) of claim 14 between at least two compatible vectors, thereby generating the selectable library.

37. The method of claim 36, wherein the recombination occurs in vitro in the presence of a site-specific recombinase.

38. The method of claim 36, wherein the recombination occurs in a cell expressing a site-specific recombinase.

39. The method of 38, wherein providing a plurality of vectors of claim 2 further comprising the steps of:
(a) introducing a plurality of the vectors into a population of prokaryotic cells;
(b) infecting a first population of prokaryotic cells with a plurality of helper phages to yield a population of phage particles; and
(c) infecting a second population of prokaryotic cells with the phage particles of (b); and optionally repeating the step of (c), thereby introducing a plurality of the vectors into a cell.

40. The method of claim 36, wherein the genetically diverse repertoire of single-chain antigen-binding unit is amenable to selection for an antigen-binding unit immunoreactive with a desired antigen in a two-hybrid system.

41. The method of claim 39, wherein the helper phage is M13 helper phage.

42. The method of claim 36, wherein the genetically diverse repertoire has a complexity ranging from 10⁶ to 10¹³.

43. The method of claim 36, wherein the genetically diverse repertoire has a complexity ranging from 10⁷ to 10⁹.

44. The method of claim 36, wherein the genetically diverse repertoire has a complexity ranging from 10⁸ to 10¹⁰.

45. The method of claim 36, wherein the genetically diverse repertoire has a complexity ranging from 10⁸ to 10¹¹.

46. The method of claim 36, wherein the genetically diverse repertoire has a complexity ranging from 10⁹ to 10¹¹.

47. The method of claim 36, wherein the genetically diverse repertoire has a complexity ranging from 10⁹ to 10¹⁰.

48. The method of claim 36, wherein the site-specific recombinase is Cre-recombinase.

49. The method of claim 36, wherein the vector of claim 14 further comprises at least two origins of replication, wherein at least one first origin facilitates replication in a prokaryotic cell, and at least one second origin facilitates replication in a eukaryotic cell.

50. The method of claim 36, wherein the second origin facilitates replication in yeast cell.

51. The method of claim 36, further comprising at least one gene encoding a selectable marker.

52. The method of claim 36, wherein the first antibody chain is a heavy chain, and the second antibody chain is a light chain.

53. The method of claim 36, wherein the first antibody chain is a light chain, and the second antibody chain is a heavy chain.

54. The method of claim 36, wherein the variable region comprises variable region sequences of a human antibody.

55. The method of claim 36, wherein the variable region comprises variable region sequences of a non-human antibody.

56. The method of claim 36, wherein the gene activation moiety comprises a transcription activation domain selected from the group consisting of GAL4 and VP16.

57. The method of claim 36, wherein the first and second site-specific recombination sequences are different sequences.

58. The method of claim 36, wherein the first or the second site-specific recombination sequence is loxP sequence.

59. The method of claim 36, wherein the first site-specific recombination sequences is loxP sequence, and the second site-specific recombination sequences is loxP2 sequence.

60. The method of claim 36, wherein the first site-specific recombination sequences is loxP2 sequence, and the second site-specific recombination sequences is loxP sequence.

61. The method of claim 36, wherein the first and/or the second site-specific recombination sequence is Frt sequence.

62. The method of claim 36, wherein the prokaryotic cell is bacterium.

63. The method of claim 62, wherein the bacterium is E. coli.

64. The method of claim 36, wherein the eukaryotic cell is a yeast cell.

65. The method of claim 36, wherein the yeast cell is S. cerevisiae.

66. The method of claim 36, further comprising a promoter 5′ to the variable region of the first antibody chain.

67. A selectable library of vectors generated by the method of claim 36.

68. A population of cells comprising the selectable library of vectors of claim 67.

69. The population of cells of claim 68 comprising yeast cells.

70. A kit comprising the vector of claim 1 or claim 14 in suitable packaging.
